(19) United States
(12) Patent Application Publication
(10) Pub. No.: US 2023/0211162 A1
(43) Pub. Date: Jul. 6, 2023

(54) Non-Invasive Peripheral Nerve Stimulation for The Enhancement of Behavioral Therapy

(71) Applicants: The Regents of the University of California, Oakland, CA (US); University of Pittsburgh - Of the Commonwealth System of Higher Education, Pittsburgh, PA (US); Board of Regents, The University of Texas System, Austin, TX (US)

(72) Inventors: Matthew Leonard, San Francisco, CA (US); Edward Chang, San Francisco, CA (US); William Schuerman, San Francisco, CA (US); Bharath Chandrasekaran, Pittsburgh, PA (US); Fernando Llanos Lucas, Austin, TX (US)

(21) Appl. No.: 18/011,117

(22) PCT Filed: Jun. 22, 2021
(86) PCT No.: PCT/US2021/038471

§ 371 Date: Dec. 16, 2022

Related U.S. Application Data
(60) Provisional application No. 63/113,509, filed on Nov. 13, 2020.
(60) Provisional application No. 63/042,423, filed on Jun. 22, 2020.

(51) Int. Cl. A61N 1/36; G09B 5/04; G09B 19/00; G09B 5/02

(52) U.S. Cl. CPC A61N 1/36092; A61N 1/36025; A61N 1/36053; A61N 1/36175; A61N 1/36157; A61N 1/36171; G09B 5/04; G09B 19/00; G09B 5/02; A61N 1/36139

(57) ABSTRACT

Systems and methods for improving behavioral therapies encompassing therapies wherein a perceptual stimulus is administered to a subject or a motor behavior is performed by the subject. Such administration of perceptual stimuli or motor performance is paired with the delivery of vagus nerve stimulation to the subject. The vagus nerve stimulation is timed with the sensory stimulus administration or motor performance in a temporal alignment that maximizes neuroplasticity and performance. Systems for performance of the method and associated software are also disclosed

### CROSS-REFERENCE TO RELATED APPLICATIONS

This application claims the benefit of priority to U.S. Provisional Patent Application Ser. No. 63/042,423, entitled “Improved Peripheral Nerve Stimulation Methods and Devices,” filed Jun. 22, 2020 and U.S. Provisional Patent Application Ser. No. 63/113,509, entitled “Improved Peripheral Nerve Stimulation Methods and Devices,” filed Nov. 13, 2020; the contents which are hereby incorporated by reference.

### STATEMENT REGARDING FEDERALLY SPONSORED RESEARCH OR DEVELOPMENT

This invention was made with government support under grant number N66001-17-2-4008, awarded by the Defense Advanced Research Projects Agency. The government has certain rights in the invention.

### BACKGROUND OF THE INVENTION

Recent neuroscience work suggests that it may be possible to overcome biological limitations on brain plasticity after developmental critical periods in adults by pairing electrical stimulation of the peripheral nervous system with behaviorally relevant events. Vagus nerve stimulation, for example, has been shown to prime adult sensory-perceptual systems towards plasticity. For example, precise temporal pairing with auditory stimuli can enhance auditory cortical representations with a high degree of specificity, for example, as described in Van Leusden, et al., 2015. Transcutaneous Vagal Nerve Stimulation (tVNS): a new neuromodulation tool in healthy humans? Front. Psychol. 6:102; Jacobs, et al., 2015. Transcutaneous vagus nerve stimulation boosts associative memory in older individuals. Neurobiol. Aging 36: 1860-1867; and Engineer, et al., 2015. Pairing Speech Sounds With Vagus Nerve Stimulation Drives Stimulus-specific Cortical Plasticity. Brain Stimulat. 8: 637-644.

Vagus nerve stimulation conveys a global diffuse signal to cholinergic and noradrenergic modulators of auditory processing, memory, and attention, for example, as described in: Shetake et al., 2012. Pairing tone trains with vagus nerve stimulation induces temporal plasticity in auditory cortex. Exp. Neurol. 233: 342-349; and Ghacibeh et al., 2006. The Influence of Vagus Nerve Stimulation on Memory. Cogn. Behav. Neurol. 19:119. Neuroimaging and animal tract-tracing studies suggest that this global neuromodulatory signal can be initiated non-invasively by applying electrical current to the auricular branch of the vagus nerve, which innervates the outer ear, for example, as described in: Badran et al., 2018. Neurophysiologic effects of transcutaneous auricular vagus nerve stimulation (taVNS) via electrical stimulation of the tragus: A concurrent taVNS/fMRI study and review. Brain Stimulat. 11: 492-500 and Frangos et al., 2015. Non-invasive Access to the Vagus Nerve Central Projections via Electrical Stimulation of the External Ear: fMRI Evidence in Humans. Brain Stimulat. 8:624-636.

The foregoing research suggests that VNS could theoretically provide a potential means of facilitating cognitive processes by its ability to activate multiple neural systems via afferent connectivity. However, to date, the practical application of peripheral neuromodulation as a means of augmenting cognitive processes has been elusive. There remains a need in the art for VNS-based protocols and tools to provide a practical benefit for subjects in the contexts of behavioral therapies, such as learning, memory, and cognitive repair. There remains a need in the art for accessible devices and methods that can translate the promise of VNS to real-world applications in the clinic and beyond.

### SUMMARY OF THE INVENTION

In a first aspect, the scope of the invention encompasses methods of enhancing the efficacy of a selected behavioral therapy, wherein the behavioral therapy comprises either a perceptual behavior or a motor behavior by the subject, the method comprising the application of VNS precisely timed to the perceptual behavior or a motor behavior. The inventors of the present disclosure have discovered that the precise temporal pairing of VNS with the presentation of a perceptual stimulus or the initiation of a motor behavior by the subject can enhance the processing of that stimulus. The inventions disclosed herein are based on the discovery that the timing of the VNS dosage administration can be temporally aligned with perceptual or motor behavior to create an optimally timed transient increase in neuroplasticity, which enhances the response of the subject to behavioral therapy. The methods of the invention provide means of enhancing learning, memorization, cognitive repair, and other cognitive abilities.

In another aspect, the scope of the invention encompasses methods of adjusting VNS dosage in response to subject performance and/or attainment of a target neural state. In one implementation the scope of the invention encompasses a closed-loop feedback system wherein subject performance or neural state is assessed and VNS dosage is adjusted according to such assessment.

In another aspect, the scope of the invention encompasses novel systems comprising devices and associated software for carrying out the methods of the invention. In one implementation, the scope of the invention encompasses a system, comprising a plurality of components, for the delivery of tVNS to a subject according to the methods of the invention. In one aspect, the scope of the invention encompasses a VNS system configured to deliver vagus nerve stimulations precisely timed to a selected behavior, to enhance the efficacy of a behavioral therapy.

The various inventions disclosed herein provide the art with a means to promote learning, cognitive repair, plasticity, and other beneficial effects. The various implementations of these inventions are described in detail next.

### BRIEF DESCRIPTION OF THE FIGURES

FIG. 1. FIG. 1 depicts the temporal alignment of tVNS administration and presentation of a Mandarin tone playback stimulus in an examplary trial of the experiments described in Example 1.

FIG. 2. FIG. 2 depicts the temporal course of neural FFR measurements and the pitch of a presented Mandarin tone stimulus, for two exemplary tones.

FIG. 3. FIG. 3 depicts the accuracy improvement (mean percentage) for trials described in Example 1 across subjects and categories for each participant group; the Generalization block (Block 7) is denoted as “GEN”. The asterisks denote statistical differences for group-by-block interactions.

FIG. 4. FIG. 4. Depicts the percent of correct trials that were retained from the previous block in the experiments described in Example 1.

FIG. 5. FIG. 5 depicts baseline and sub-threshold vagal evoked potentials (mean) for participants receiving stimulation in the experiments of Example 1. The three significant evoked potentials are denoted as N1, P1, and N2.

FIGS. 6A, 6B, and 6C. FIGS. 6A, 6B, and 6C depict performance for three subjects in a word learning task where native English speakers were tasked with associating Mandarin words with objects, as described in Example 2.

FIG. 7. FIG. 7 depicts a tVNS stimulation setup of the invention, wherein the tVNS electrodes are held in place by putty molded to the individual subjects' ear. In this implementation, the anode is applied to cymba concha and the cathode is applied to the cymba cavum.

FIG. 8. FIG. 8 is a diagrammatic overview of system depicting elements of an exemplary system, including: a computing system in connection with the elements (depicted by pointing arrows); a timing controller; pulse generator; VNS electrode assembly; brainwave monitoring device; camera, microphone, speaker, display screen, and keyboard.

FIG. 9. FIG. 9 is a graphical depiction of the timing of VNS administration and perceptual stimulus administration or performance of motor behavior in a temporal alignment wherein the VNS administration precedes the perceptual stimulus administration or motor performance by an offset amount, and overlaps with the perceptual stimulus administration or motor performance by an overlap amount.

### DETAILED DESCRIPTION OF THE INVENTION

In a general implementation, the scope of the invention encompasses a method of enhancing the efficacy a behavioral therapy in a subject,
wherein the behavioral therapy comprises a perceptual or motor behavior by the subject;
the method comprising the administration to the subject of vagus nerve stimulation;
wherein the vagus nerve stimulation is administered in a temporal alignment with the motor or perceptual behavior that is selected to enhance the efficacy of the behavioral therapy.

In one implementation, the scope of the invention encompasses a method of enhancing the efficacy a behavioral therapy associated with perception of a perceptual stimulus by the subject;
the method comprising the administration to the subject of the perceptual stimulus; and
the administration to the subject of vagus nerve stimulation;
wherein the vagus nerve stimulation is administered in a temporal alignment with the administration to the subject of the perceptual stimulus that is selected to enhance the efficacy of the behavioral therapy.

In another implementation of the invention, the scope of the invention encompasses a method of enhancing the efficacy a behavioral therapy comprising the performance of a motor behavior by the subject;
the method comprising the administration to the subject of vagus nerve stimulation;
wherein the vagus nerve stimulation is administered in a temporal alignment with the performance of the motor behavior by the subject that is selected to enhance the efficacy of the behavioral therapy.

The various implementations and elements of these methods are described next.

Subjects. The method of the invention are applied to a subject. In a primary embodiment, the subject is a human subject. In one context, the subject may be a subject seeking to learn a new ability, reinforce a previously learned ability, or augment or improve an ability. For example, in one embodiment the subject is learning a non-native language. In other contexts, the subject may be a patient or other person in need of treatment for a cognitive deficit or impairment. Exemplary subjects may include subjects with speech impediments, motor deficits, behavioral disorder, or other cognitive deficits. In some implementations, the subject is a subject in need of treatment for a cognitive impairment, such as a subject that has undergone brain surgery, traumatic brain injury, neurodegeneration, brain cancer, epilepsy, ischemia, or other injury. In some embodiments, the subject is an aged subject, for example, a subject of at least forty, at least forty five, over fifty, over fifty five, or over sixty years of age.

In an alternative implementation, the subject may comprise a non-human animal. Exemplary non-human subjects include test animals, animal models of disease or aging, livestock, veterinary subjects, or pets. Exemplary non-human subjects include non-human primates, rodents, canines, felines, bovines and equines.

Behavioral Therapies. The scope of the invention is directed to the enhancement of the efficacy of a selected behavioral therapy. Enhancement may encompass any measurable improvement in performance, such as an increase or improvement in: ability, speed, accuracy, retention, acquisition, or restoration of a cognitive or function, or other outcome of the selected behavioral therapy. For example, in one implementation, improvement is assessed by comparison of the performance by subjects wherein the methods of the invention have been applied with the performance of subjects wherein methods of the invention were not applied.

The behavioral therapy may comprise any behavioral therapy known in the art. In one embodiment, the behavioral therapy is a behavioral therapy associated with the receipt of a perceptual stimulus by the subject. Associated with, in this context, may mean that receipt of the perceptual stimulus is utilized to train, teach, or otherwise promote the outcome of a behavioral therapy. Accordingly, in one aspect, the methods of the invention encompass the administration of a perceptual stimulus to the subject, wherein administration is timed with the delivery of VNS in a selected temporal alignment.

In some implementations, the perceptual stimulus comprises an auditory stimulus, such as a sound, a noise, a note, a tone, etc. In one embodiment the auditory stimulus is hearing a non-native language phoneme, word, or phrase.

The perceptual stimulus may comprise a visual stimulus, such as viewing a physical object or scene, or viewing a visual representation thereof. The visual stimulus may comprise viewing a symbol. In some embodiments, the visual stimulus may be configured as a photograph, drawing, animation, or video.

The perceptual stimulus may comprise a tactile stimulus. Exemplary tactile stimuli include, for example, neural feedback generated by the performance of a movement; interaction with, e.g., handling of, a physical object; or application of forces to the subject by a physical therapy device, training device, or by a therapist.

The perceptual stimulus may comprise other types of stimuli. For example, an olfactory stimulus, for example, a taste or smell of a substance. Other types of perception that may be addressed include subliminal perception. For example, in the visual modality, subliminal perception is controlled with stimulus presentation time (e.g. the picture/word is presented for less than ˜20 ms to create a subliminal perception). In the auditory modality, subliminal perception is controlled with noise (e.g., adding noise to the target speech or auditory signal) or attention (e.g., incidental learning).

In some implementations, the administered stimulus may comprise a combination of stimuli. For example, a subject viewing a video with sound will be presented simultaneously with visual and audio stimuli. For example, a subject in a flight simulator may be presented with the tactile stimuli of the instruments she or he is manually engaging with as well as the visual stimulus of a simulated cockpit window.

Exemplary behavioral therapies associated with receipt of a sensory stimuli may include, for example, various types of learning, memorization, and cognitive repair. Learning, for example, may encompass any acquisition of knowledge or skill. Learning may encompass, in various embodiments: recognition of objects or symbols; recognition of sounds or words; recognition of concepts; and acquisition of the ability to perform a task. In one implementation, the behavioral therapy is learning and encompasses the learning of a non-native language, e.g. a language that the subject did not previously learn. In one embodiment, the behavioral therapy encompasses auditory learning of words, phrases, tones, or phonemes. In one embodiment, the behavioral therapy encompasses learning to recognize written words or symbols of the non-native language by visual perception thereof. In one embodiment, the therapy encompasses processing and production of language-specific structures, as captured by the phonology, morphology, syntax, semantics, pragmatics of a target language, for example in the context of restoring language-specific skills in various types of dementia and neurodegenerative disorders affecting language.

In one implementation, the behavioral therapy is memorization. Memorization may encompass any encoding, storage, and retrieval of information in the brain. In various embodiments, the memorization may encompass short-term memory, long-term memory, episodic memory, semantic memory, implicit memory, or procedural memory.

Memorization may encompass memorization of information comprising order, meaning, temporal, or spatial aspects. Other aspects of memory that may be improved by therapy include working memory and emotional memory.

In one implementation, the behavioral therapy is cognitive repair. Cognitive repair, or cognitive rehabilitation, may encompass the reinstatement of a cognitive ability by restoration of associated neural networks or the development of compensatory neural networks. Cognitive repair may occur in the context of any impairment, for example, impairment from trauma and injury, disease, neurodegenerative disorder, aging or other causes. In one embodiment the therapy comprises restoration of language skills in any sensory modality (e.g., spoken, sign, or haptic), for example, restoration after vascular or neurodegenerative disease.

Motor Behaviors. In one implementation, the behavioral therapy encompasses a motor behavior. In this implementation of the invention, VNS administration is timed to the subject's performance of the selected motor behavior, with the temporal alignment of the VNS and motor behavior performance being selected to enhance the subject's performance. A motor behavior may encompass any performance of a specified movement or combination of movements by the subject. The behavioral therapy may encompass any therapy associated with performance of particular motor functions by the subject, in various contexts being applied to assist the subject in learning, augmenting, or restoring a selected motor skill. Motor therapies may encompass, for example, speech therapy, physical therapy, or any other motor therapy, for example, directed to fine motor skills, gross motor skills, motor planning, and other such therapies.

In one implementation, the motor therapy is a rehabilitative therapy, i.e. helping to restore motor functions that have been compromised in subjects by injury, disease, neurodegeneration, or aging. In a related context, the motor therapy encompasses teaching or augmenting motor activities in otherwise impaired subjects, e.g. subject with developmental disorders.

Motor therapies may encompass any number of motor activities, for example, movements made in the course of daily living (e.g. walking, eating, dressing, grooming, etc., for example, in a rehabilitative context), movements made in the operation of devices, implements, or machinery (i.e. operating vehicles, use of surgical tools), movements made in sport; or movements made in the playing of a musical instrument.

In one implementation, the motor functions are associated with speaking a language, for example pharyngeal, lingual, or other complex movement combinations requiring in voicing of words, phonemes, or phrases. In one embodiment, the motor therapy is applied in the learning of a novel (non-native) language. In one embodiment, the motor therapy is speech therapy for speech impaired individuals having difficulty or impairment in speaking a language. In one embodiment, the motor therapy is rehabilitative speech therapy for subjects that have suffered cognitive injury, disease, or aging or other impairments.

VNS Administration. The scope of the invention encompasses the administration of a VNS dosage to a subject. As used herein, VNS means the electrical stimulation of the vagus nerve to create a transient state of neuroplasticity. The vagus nerve, as known in the art, is designated as the tenth cranial nerve and may be denoted as “CN X.” The vagus nerve has both sensory and motor functions, and is implicated in parasympathetic control of pulmonary, cardiac, and digestive functions. The vagus nerve comprises a substantial number of afferent nerves conveying sensory signals from various organs and parts of the body to the central nervous system. The vagus nerve has left and right side branches that descend from the cranial vault through the jugular foramina and branch throughout the body.

It is known in the art that certain electrical stimulations of the vagus nerve will deliver an afferent signal to the brainstem, resulting in a transient release of various neurotransmitters. Suitable stimulation of the vagus nerve will induce the release of norepinephrine (NE, also known as noradrenaline) by the locus coeruleus as well as the release of gamma-aminobutyric acid (GABA), acetylcholine (ACh), serotonin, and other neurotransmitters. It is believed in the art that this release of neurotransmitters creates a transient state of neuroplasticity, i.e. a state favorable for the growth, reorganization, and reinforcement of neural networks in the brain.

VNS Application Sites. The stimulation may be achieved by various devices and routes of administration. In one implementation, the stimulation is achieved by an implanted device, such stimulation being known as invasive vagus nerve stimulation or iVNS. iVNS devices are known in the art and have been clinically deployed in the treatment of epilepsy, depression, and pain management. Exemplary devices comprise a pulse generator, typically implanted in the chest. A subcutaneously implanted electrical lead connects the pulse generator to electrodes that are placed on or around the left branch of the vagus nerve at a site in the neck. Exemplary commercial iVNS systems include, for example, DEMIPULSE™ and ASPIREHC™ devices (Livallova USA, Inc., Houston, Tex., US) utilized in the treatment of epilepsy; VNS THERAPY™ and CYBERONIC PROSTHESIS™ systems (Cyberonics, Houston, Tex., US) for treatment of arthritis and epilepsy.

In a primary embodiment, the VNS stimulation is achieved by transcutaneous vagus nerve stimulation (tVNS). tVNS encompasses the stimulation of the vagus nerve by energy applied to the body externally, advantageously avoiding the complications and trauma of an implanted device. tVNS devices may be applied at any site wherein a branch of the vagus nerve is accessible.

In a primary embodiment, tVNS is applied to the ear to stimulate the auricular branch of the vagus nerve. Cutaneous afferent vagus nerves fibers are found in various structures of the ear, including the anterior wall of the ear canal, the antihelix, the tragus, the concha, and the cymba concha of the ear, providing convenient sites for the application of electrical signals to stimulate the vagus nerve. In an alternative implementation, tVNS is administered to the cervical branch of the vagus nerve at sites on the neck.

VNS may be applied to the left branch of the vagus nerve. In some embodiments, the VNS is administered to the right branch of the vagus nerve. In one embodiment, both the left and right branches of the vagus nerve are stimulated.

VNS Devices. VNS may be applied by the use of electrodes in connection with pulse generating devices by leads (wires), for example, bipolar leads. Exemplary electrodes may comprise any conductive material, e.g. metals such as silver, titanium, stainless steel, and alloys of the foregoing. Exemplary electrodes include silver and AgCl electrodes. Electrodes for VNS may comprise wires, for example, helical wires configured to encircle the vagus nerve, as in iVNS systems. Electrodes for tVNS systems may comprise, for example, disks, rectangular bodies, ovoids, or arrays comprising multiple contact points. Exemplary electrodes include disks, for example, disks of 1-5 mm in diameter, for example, about 4 mm in diameter.

Electrodes of the VNS application device will generally comprise an anode and a cathode, which may be placed in proximity to each other. In one embodiment, the anode is applied to a site innervated by fibers of the vagus nerve. In one embodiment, the cathode is applied to a site innervated by fibers of the vagus nerve. In one embodiment, both the anode and cathode are applied to sites inervated by the vagus nerve. For example, in one embodiment, the anode is applied to cymba concha and the cathode is applied to the cymba cavum. In one embodiment one or both of the anode and cathode are applied to the ear canal.

The electrodes may be present in an assembly that is worn or otherwise held in place in a target anatomical region of the subject. In one embodiment, the assembly comprises a conformable substance (e.g. polymeric putty or like material) molded to the ear of the individual subject. In one embodiment, the electrodes are placed in the conformable substance such that when the assembly is deployed to the subject, the electrodes are in stable contact with target areas. In one embodiment, the assembly comprises a silicone body molded to fit the ear, for example, a generic body or one customized to the ear of the subject. in one embodiment, the electrode assembly comprises an earbud or earphone that fits in the ear canal. In one embodiment, the electrodes are held on the skin at the target sites by an adhesive material, or are held in place by medical tape. In one embodiment, the assembly comprises a spring-loaded clip that holds the electrodes in place on the ear or other site of application. In one embodiment, the assembly comprises a collar that holds electrodes in place on the neck.

The VNS application device will comprise a pulse generator. The pulse generator comprises one or more devices that generate electrical impulses with precisely tailored dosage parameters, as described below. The pulse generator may comprise a computer controlled potentiostat with fine tunability of electrical outputs.

Various commercially available tVNS systems are known in the art. Exemplary systems include the GAMACORE™ device (Electrocore systems, Rockaway N.J., US), the CERBOMED™ device (Nemos, Erlangen, Del.), and the NMS 300™ device (Xavant Technology, Silverton, SA).

When applied to the subject, the electrodes may be covered with a conductive gel such as a salt-free conductive gel. Exemplary conductive gels include polymeric materials such as glycerin, glycols, maleic anhydride, vinyl ethers, and other compositions known in the art.

VNS Dosage Parameters. The VNS will be applied in a therapeutically effective amount, meaning an amount sufficient to produce a measurable transient release of neurotransmitters such as NE in the brain. In one embodiment, the therapeutically effective amount is an amount sufficient to induce a measurable change in vagus sensory evoked potential (VSEP), which may be used as a neurophysiological indicator of vagus nerve activity. The VNS dosage comprises electrical energy pulses having various properties.

A first parameter of the VNS dosage is the waveform of the applied energy. Any suitable waveform sufficient to induce neurotransmitter release may be used. Exemplary waveforms include monophasic waveforms and biphasic waveforms, for example square or rectangular waveforms. Sinusidal waveforms may also be utilized. In a primary embodiment, the waveform will comprise a charge-balanced waveform. In one embodiment, the waveform is a biphasic square waveform, as known in the art. The pulse width may comprises any length, for example in the range of 10-1,000 μs, for example 50-200 μs, for example about 150 μs.

A second parameter of the VNS dosage is the amplitude of the applied energy, also referred to as intensity. Amplitude refers to the magnitude of the applied current. Exemplary tVNS applications may be in the range of 0.1-15 mA, for example, 0.5 to 10 mA, for example, in the range of 1.0-2.0 mA. As observed previously and described herein, brain response to VNS does not linearly increase with stimulation intensity. In some cases, the relationship between tVNS intensity and the pulse-evoked brainstem response follows a non-linear, inverted-U pattern with stronger engagement at intermediate VNS intensities (for example, 1.0 mA to 2.0 mA), as compared to low (0.2 mA to 1.0 mA) and high (2.0 mA or greater) intensities. Accordingly, the applied tVNS dosage intensity in some embodiments may be selected at a value within the intermediate range (1.0 mA to 2.0 mA) for optimized effect. For iVNS systems, much less energy is required to achieve the desired biological effect, and the intensity of the applied energy may be, for example, in the range of 0.1-0.5 mA.

In a primary implementation, the methods of the invention encompass the administration of subperceptual VNS. Subperceptual VNS is advantageously better tolerated and more comfortable for subjects, and does not distract subjects from the learning, rehabilitative, or other tasks performed in the methods of the invention. Exemplary subperceptual tVNS current values may be in the range of 0.1-5.0 mA. In one implementation, subperceptual tVNS intensities may be determined by assessing an individual subjects' perceptual threshold of tVNS application by means known in the art. For example, a staircase procedure may be utilized across selected current, frequency or other parameters to determine the perceptual threshold of the putative VNS treatment in the subject. A subperceptual application is any administration below this determined threshold. For example, the selected subperceptual application may be at a selected magnitude below the determined perceptual threshold of the subject, for example, 0.1 mA, 0.15 mA, 0.2 mA, 0.25 mA, or 0.3 mA below the perceptual threshold of the subject. Exemplary subperceptual thresholds observed by the inventors of the present disclosure range from 0.1 to 1.3 mA, with the variability believed to be due in part to differences in skin conduction properties between subjects. Subperceptual thresholds will also be affected by stimulation frequency and pulse width values, which may be adjusted accordingly.

Another VNS dosage parameter is the frequency of the applied pulses. VNS pulse frequency may be applied at any desired frequency that imparts the desired biological effect while avoiding harm. In various embodiments, the frequency may be, for example, 1-300 Hz, for example, 5-30 Hz, for example, about 8-12 or about 20-30 Hz.

In one embodiment, the VNS dosage applied in the methods of the invention has the following parameters: a transcutaneous VNS applied as 10-20 biphasic square-wave pulses having pulse width of 100-200 μs, applied at a frequency of 25 Hz, with an intensity between 1.0 mA and 5.0 mA, and/or selected to be 0.1-2.5 mA below the subject' perceptual threshold.

It will be understood that the optimal intensity range of intensities for brain activation may differ across VNS delivery modalities, and one of skill in the art may adapt the methods disclosed herein to various tVNS application systems having different stimulation sites, configurations, and other properties. Other considerations include the co-administration of VNS with other brain modulating agents or treatments, for example, VNS in combination with drugs (e.g., reuptake inhibitors) and other brain stimulation techniques (e.g., transcranial magnetic stimulation or direct current stimulation) to enhance motor and perceptual therapy.

Temporal Pairing of VNS Administration to Subject Behavior. The inventors of the present disclosure have advantageously determined that in behavioral therapy accompanied by administration of a VNS dosage, the delivery of the VNS dosage can be timed with perceptual or motor behavior in various ways to provide enhanced outcomes.

The administration of the VNS dosage will comprise delivery of a series of electrical pulses to the vagus nerve during an interval of time referred to herein as the “VNS window.” The window is defined by a VNS starting point, being the time at which VNS delivery commences and a VNS endpoint, being the at which delivery is completed.

In the case of behavioral therapies comprising administration of a perceptual stimulus to the subject, the “stimulus window” will refer to an interval of time during which the stimulus is administered to the subject. The stimulus window comprises a stimulus starting point, being a time point at which administration commences and a stimulus endpoint, being at time at which stimulus administration is complete. For example, in the case of an audio stimulus comprising a recording of a word, the time elapsed in playback of the recorded word will be the stimulus window.

In the case of behavioral therapies comprising performance of a motor behavior by the subject, the “motor window” will refer to an interval of time during which the motor behavior is performed by the subject. The motor window comprises a motor starting point comprising the time at which performance of the motor behavior commences and a motor endpoint comprising the time at which performance of the motor behavior is complete.

This definitional framework provides a means to describe the temporal alignment of the VNS window and the stimulation window.

In a first embodiment, the VNS window starting point is prior to the stimulus staring point or motor starting point, as the case may be, wherein the time interval between the VNS starting point and the stimulus starting point, or the difference between the VNS starting point and the motor starting point, are termed herein as an “offset.”

The inventors of the present disclosure have determined that an offset between the VNS window and the behavior window, i.e. the stimulus window or motor window, provides a substantial enhancement of the efficacy of the behavioral therapy. Without being bound to any particular theory of mode of action, it is believed that the administration of the VNS just prior to the perceptual or motor behavior induces an optimized state of neural plasticity that enhances the formation, reorganization, and reinforcement of neural networks associated with the selected behavioral therapy. For example, a working demonstration is provided herein in the Examples section below, wherein the auditory recognition of non-native language phonemes was significantly enhanced by administration of VNS at a time commencing just prior to presentation of an audio stimulus comprising the sounds to be learned.

In some embodiments, the VNS offset comprises from 0.001 to 10.0 seconds prior to the administration of the perceptual stimulus, for example, 10 ms to 2.0 seconds. In some embodiments, offset comprises 50-1,000 ms In some embodiments, the offset comprises about 100-500 ms, in various embodiments the VNS starting point being about 100, 125,150, 175, 200, 225, 250, 275, 300, 325, 350, 375, 400, 425, 450, 475 or 500 ms prior to the stimulus starting point or motor starting point.

In a primary implementation, the VNS window overlaps an initial portion of the behavior window, i.e. the stimulus window or motor window, wherein the endpoint of the VNS window is during the behavior window. This overlap may comprise any time interval, for example, wherein the VNS window of administration occurs over 100-1,000 ms the stimulus or motor window, for example, in various embodiments, the overlap being about 100, 125,150, 175, 200, 225, 250, 275, 300, 325, 350, 375, 400, 425, 450, 475 or 500 ms.

This overlap may comprise any proportion of the behavior window. The duration of the sensory behavior window will vary depending on the context of the behavioral therapy. For example, an auditory stimulus comprising a syllable may have a duration of about 100-800 ms, for example 300-500 ms. Other stimuli may require multiple seconds to be administered and/or perceived and processed. Motor behavior movements will vary in duration as well, for example, in the range of 10 ms to several seconds. Accordingly, to account for variability of behavior window duration, the overlap of the VNS administration window with the behavior window may be described according to the proportion of the behavior window it overlaps, for example, on a scale of 0-100% of such duration. In various embodiments, the selected VNS endpoint is timed to be about or at least 5%, 10%, 15%, 20%, 25%, 30%, 35%, 40%, 45%, 50%, 55%, 60%, 65%, 70%, 75%, 80%, 85%, 90% 95%, or 100% through the duration of the behavior window. For example, in one implementation, the VNS administration endpoint is timed to a point at about 25-75% through the duration of the behavior window, for example, at a time point at least about 50% through the duration of the stimulus or motor window, as the case may be.

in some implementations, the VNS window continues past the endpoint of the behavior window, for example, in the context of short behavior windows. The lag between the behavior endpoint and VNS endpoint may be of any duration, for example, in the range of 10-1,000 ms or longer, in various embodiments being about 50, 100, 150, 200, 250, 300, 400 or 500 ms.

In alternative implementations, the VNS starting point lags behind the behavior window starting point, for example, being at a time point after the stimulus starting point or being at a time point after the motor starting point, as the case may be. Such an implementation misses the “priming” effect of the offset described above, however may will generally still provide enhancement of the behavioral therapy. In these implementations, the lag time interval between the behavior starting point and the VNS starting point may be any time, for example, in the range of 10-500 ms, for example, in various embodiments being about 10, 50, 75, 100, 150, 200, 250, 300, 350, 400, 450, or 500 ms.

In another alternative implementation, the VNS window follows the behavior window, i.e. the VNS starting point is a time point after the behavior window, i.e. stimulus window or motor window endpoint.

The alignment of the VNS administration with the behavior is achieved by systems of the invention, as described below. In the case of behavioral therapies comprising the administration of a perceptual stimulus, the systems will be configured to pair such delivery with VNS administration, for example, with millisecond-scale resolution. In a general embodiment, the system of the invention is configured to deliver the VNS-stimulus combination with temporal alignment selected as described above. In one embodiment, the combined delivery is administered in response to a user request. In this implementation, the system is configured to receive a request input from a user, wherein upon receipt of such input, the combined delivery is initiated. In various exemplary embodiments, the input may be any of the following: the subject can click a displayed button on a screen or touchscreen; the subject can enter a keystroke on a keyboard; the subject may issue a verbal command; the subject may engage a button, switch or other mechanoelectrical device that signals the system to initiate the combined delivery. Alternatively, a therapist or attendant may enter the request. Alternatively, the a series of combined deliveries are administered according to a predetermined sequence (e.g. one visual or audio stimulus is displayed or played every five seconds, etc.).

In the case of a behavior comprising an administered stimulus as described above, both the stimulus delivery and the VNS administration are controlled by the system. However, in the case of a behavioral therapy comprising a motor activity, an added layer of complexity is introduced, because the VNS administration must be timed to a behavior that is initiated and performed by the subject. Thus, the system must detect or predict the timing of the motor behavior in order to pair the VNS administration therewith.

In one implementation, the motor behavior is detected by elements of the system. For example, automated systems comprising cameras, motion sensors, such as ultrasonic or infrared motion sensors, accelerometers or other elements worn, engaged with, or held by the subject may be used. Motion detection and analysis systems known in the art may be used, such as those described in U.S. Ser. No. 10/157,488; U.S. Pat. Nos. 9,401,178 and 9,682,280.

in the case of motor behavior detection, the administration of the VNS may have to administered after the onset of the behavior. Although this timing misses the advantageous priming effect of the VNS window offset described above, administration during the behavior will still provide neuroplastic benefit and enhancement of the motor behavior.

Alternatively, the system can be configured to predict the timing of the initiation of the motor behavior. In one embodiment, the system is configured to issue a prompt to the subject to initiate the motor behavior, for example, playing of a tone or display of a prompt on a screen. Following issuance of the prompt, the user will initiate performance of the motor behavior. The delay between prompt issuance and motor behavior initiation will comprise a time interval or latency. By testing multiple subjects with the same system and task, for example, using the task detection functions described above, a probability distribution of latency time intervals can be determined for the behavior and used to predict the onset time of the motor behavior initiation, with VNS administration timing based on the predicted latency. Alternatively, the latency can be determined on a subject-by-subject basis, with a calibration session comprising multiple trials used to determine the timing between prompt issuance and initiation of motor behavior for that individual subject. This data can be used to predict the timing of motor behavior initiation and can be used to time VNS administration.

In another implementation, the timing of motor behavior onset is predicted by the system using pre-movement cognitive or motor cues, measured by appropriate detection elements. For example, EEG, EcoG, EMG or other detection elements of the system may be used monitor the subject for cues that precede onset of the selected motor behavior. Various motor intention prediction tools are known in the art, for example, as described in Luzheng et al., 2019. A review on EMG-based motor intention prediction of continuous human upper limb motion for human-robot collaboration. Biomedical Signal Processing and Control 51: 113-127; Kang et al., 2018. Prediction of movement intention using connectivity within motor-related network: An electrocorticography study. PLoS ONE 13: e0191480; and Gowrishankar et al., 2018. Utilizing sensory prediction errors for movement intention decoding: A new methodology. Science Advances 4: eaaq0183 DOI. For each subject, a calibration session may be performed with several trials to determine individual subject cues that reliably predict the initiation of the motor behavior. Thereafter, detection of the cues is utilized to time VNS administration and enables administration prior to the initiation of the motor behavior.

Feedback-Controlled VNS Dosage Modulation. In some implementations of the methods of the invention, the VNS dosage is adjusted based on the subject's performance or neural state. For example, in some implementations, the subject is evaluated on their performance of a task, such as recognizing a sound or symbol, voicing a sound or word, or performing a motor task. In a primary embodiment, such performance monitoring and evaluation is achieved in an automated manner by suitable devices, including, for example, speech recognition software, movement analysis systems, e.g. comprising video capture of subject movement; feedback from therapeutic devices handled by the subject, etc. Alternatively, the monitoring and evaluation can be performed manually, for example by a therapist or testing attendant, however such manual implementations will be slower than automated systems.

In another implementation, the attainment of a selected target neural state will be used to guide VNS dosage. Neural state may comprise, for example, desynchronization of low frequency oscillatory activity in the cortex. In one embodiment, the neural state is VSEP above a selected threshold value. Evaluation of neural state may be achieved by any suitable device or tool. In some implementations, neural state is assessed by electromyography (EMG), electroencephalogram (EEG) or electrocorticography (ECoG).

Modulation of VNS dosage in response to feedback may encompass changing the magnitude of the applied dosage, or the timing thereof. In one aspect, the scope of the invention encompasses a method of titrating VNS dosages, wherein the delivered dosage is sequentially altered by a selected amount over a course of therapy or treatment. In one embodiment, the dosage is titrated downward, for example, beginning at a higher relative value and being reduced over time, for example, as the participant becomes more proficient at a selected task. Alternatively, the dosage could be increased over time, for example, to push cognitive enhancement further. Upward or downward adjustments may comprise may, for example sequential increases or reductions of 5%, 10%, 15%, 20%, or 25% of the initial dosage value.

In one embodiment, a VNS dosage is administered after evaluation of subject performance or neural states. In this implementation, it is determined whether or not the subject successfully achieves a goal or if the target neural state was attained. If so a VNS dosage is administered to reinforce the behavior, and if the subject does not successfully perform the task or achieve the selected neural state, VNS is not administered.

Systems of the Invention. The scope of the invention further encompasses systems for performing the methods disclosed herein. A system, as used herein, comprises two or more elements used in combination to achieve a selected method of the invention. In the case of a behavior therapy associated with the administration of a perceptual stimulus, a general system of the invention comprises:

one or more devices for the administration of the selected perceptual stimulus to a subject;
one or more devices for the administration of a VNS dosage to the subject; and
one or more control elements that time the VNS administration window to the stimulus window by a selected temporal alignment.
   In the case of behavior therapy comprising the performance of a motor behavior by the subject, a general system of the invention comprises:
one or more devices for detecting, or predicting the timing of, the initiation of the motor behavior;
one or more devices for the administration of a VNS dosage the subject; and
one or more control elements that time the VNS administration window to the detected or predicted timing of the motor behavior window by a selected temporal alignment.
   In one embodiment, the scope of the invention encompasses a system for use in a method of enhancing the efficacy of a behavioral therapy in a subject, the system comprising: one or more devices for the administration of VNS to the subject; and one or more control elements that time the administration of VNS to the subject to a perceptual behavior or motor behavior with a selected temporal alignment.
   In one embodiment, the scope of the invention encompasses a system for use in a method of enhancing the efficacy of a behavioral therapy in a subject, wherein the behavioral therapy comprises administration of a perceptual stimulus, the system comprising: one or more devices for the administration of the selected perceptual stimulus to a subject; one or more devices for the administration of a VNS dosage to the subject; and one or more control elements time the VNS administration window to the administration of the perceptual stimulus by a selected temporal alignment.
   In another embodiment, the scope of the invention encompasses a system for use in a method of enhancing the efficacy of a behavioral therapy in a subject, wherein the behavioral therapy comprises the performance of a motor behavior by the subject; the system comprising: one or more devices for detecting, or predicting the timing of, the initiation of a motor behavior; one or more devices for the administration of a VNS dosage the subject; and one or more control elements that time the VNS administration to the detected or predicted timing of the motor behavior by a selected temporal alignment.

The device(s) for the administration of a selected perceptual stimulus to a subject will be selected based on the particular stimulus to be applied. For example, an audio stimulus may be administered by an electronic speaker, i.e. a device that transduces electromagnetic signals into sound waves. A visual stimulus may be administered by a video display, i.e. a screen. Alternatively, the visual stimulus may be administered by presentation of a printed picture or presentation of a physical object or scene. A tactile stimulus may be administered by a device having mechanical elements that apply forces to the subject or which are handled by the subject or are otherwise in contact with the subject. The tactile stimulus may be administered by a human, such as a physical therapist.

Other system elements may include elements for detection or evaluation of user movements, such as video cameras, motion sensors, accelerometers, inertial sensors and others. Elements for recording subject voice may include microphones. Elements for issuing prompts will include speakers and/or display screens. Elements for receiving user inputs may include graphical user interfaces, touchscreens, and others. Elements for assessing behavioral performance or neural state of the subject, evaluating subject performance, and predicting motor behavior may include tools for the measurement of brain function or other physiological parameters, for example, EMG, EEG, or ECoG devices applied to the subject.

As described above, the one or more devices for the administration of VNS may comprise any VNS delivery system known in the art, for example an iVNS system or a tVNS system, for example an auricular or cervical tVNS system.

The functions of the system and integration of the inputs and outputs will be achieve by one or more computing devices and associated software. In one embodiment, the system of the invention encompasses a general purpose computing device, e.g. a microprocessor, distributed computer system, cloud computing system, or other computing device or system. The computing system will be operated by associated software, e.g. non-transitory storage medium comprising a set of computer-readable instructions which operate the elements of the system carry out the steps of the methods disclosed herein. The one or more control elements will be in functional connection with the various elements of the system as configured. Such connections may be wired or wireless.

A key element of the system is an element that will be referred to herein as the timing controller. The timing controller comprises one or more control elements that align the timing of the VNS window to the behavior window. The timing controller may comprise software that directs the precise timing of VNS administration by associated hardware elements, for example, with millisecond resolution, and will rely on execution by hardware elements having the requisite ability to resolve VNS pulse administration and other functions with the selected resolution. An exemplary technique to provide the required resolution includes, for example, buffering pulse waveforms prior to delivery. Other techniques for precise alignment may be adapted from methods known in the art, for example, as described in Asaad and Eskander, 2008. Achieving behavioral control with millisecond resolution in a high-level programming environment, Journal of Neuroscience Methods 173: 235-240.

The controller elements may be configured to deliver the perceptual stimulus in response to an input from the subject, an input from a therapist, or according to a pre-programmed sequence.

The systems of the invention may comprise any other physical elements, such as computers, servers, data storage or computer memory, network connections, video screens, mobile devices, keyboards, touchscreens, power supplies, and other components necessary for operation of the system.

### EXAMPLES

### Example 1. Non-Invasive Peripheral Nerve Stimulation Selectively Enhances Speech Category Learning in Adults

Here was examined the impact of tVNS on the acquisition of new speech categories in adulthood. tVNS was paired with non-native speech stimuli in a speech category training task. Native English-speaking adults were trained to categorize acoustically different Mandarin Chinese syllables into four Mandarin tone categories as a function of their pitch contour. Mandarin Chinese has four non-neutral syllabic pitch contours (i.e., tones) that change word meaning and are lexically irrelevant in English: high-level (“Tone 1”), low-rising (“Tone 2”), low-dipping (“Tone 3”), and high-falling (“Tone 4”) tones. While Mandarin tones are acoustically distinguishable by relative differences in pitch height and pitch direction, English-speaking learners are perceptually more sensitive to relative differences in pitch height. Tone 1 and Tone 3 are acoustically cued by higher and lower pitch values, respectively, and are therefore perceptually more salient for English learners. Thus, for English learners, Tone 1 and Tone 3 are easier-to-learn than Tone 2 and Tone 4.

Based on this distinction between easier- vs. harder-to-learn tone categories, participants were split into two experimental groups that received paired tVNS with either Tone 1 and Tone 3, or with Tone 2 and Tone 4. Stimulation intensity was delivered below the perceptual threshold of each learner. the performance of these two experimental groups was compared with a control group of learners that did not receive stimulation during training. This experimental manipulation allowed assessment the specificity and extent of generalization in VNS-related behavior and auditory sensory plasticity.

To rule out pre-training differences in perceptual identification skills and auditory sensory encoding between groups, a perceptual identification task was performed and scalp-recorded frequency-following responses (FFRs) were collected before the training session. FFRs were also collected after the training session to assess the extent to which tVNS modulated the sensory representation of non-native pitch. Additionally, electrophysiological correlates of VNS were collected in every participant receiving stimulation to assess the extent to which sub-threshold peripheral nerve stimulation evoked brainstem activity supportive of peripheral nerve engagement.

On this short timescale, tVNS did not modulate the sensory representation of Mandarin tones, as measured by the FFR. These results demonstrate that it is possible to enhance adult speech category learning in a highly specific manner by inducing a temporally precise neuromodulatory signal via non-invasive peripheral nerve stimulation.

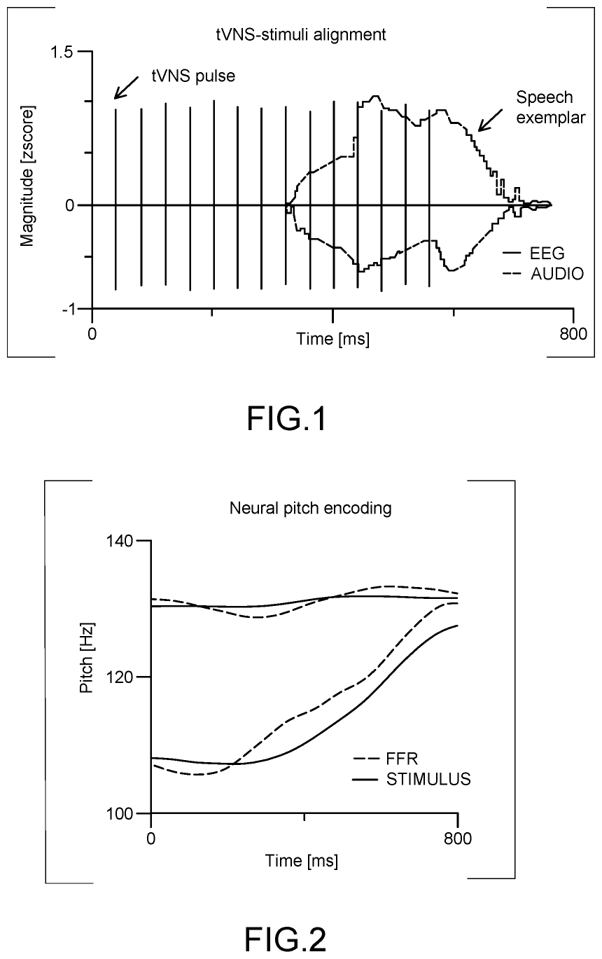

36 native English speakers were trained to categorize natural speech exemplars of the four Mandarin tone categories. Stimuli were presented in six training blocks, and each tone exemplar was presented once per block. On each trial, participants indicated which category they heard and received visual feedback (“Correct”/“Wrong”) following their response. Stimulation intensity was delivered below the perceptual threshold, surrounding the onset of the auditory stimuli, as depicted in FIG. 1. Sub-perceptual stimulation thresholds were calibrated on an individual participant basis, using a staircase procedure. The two stimulation groups differed most when tVNS was paired with the tone categories that were easier-to-learn (Tone 1 and Tone 3; “tVNS-easy group”) or harder-to-learn (Tone 2 and Tone 4; “tVNS-hard group”). These tone categories were selected on an empirical basis, based on a cohort of 678 English learners of Mandarin tones (“Aggregate dataset”) collected across eight published studies using no stimulation. The analysis of correct responses by category in the Aggregate dataset revealed that Tone 1 and Tone 3 were easier-to-learn than Tone 2 and Tone 4 (one-way ANOVA: F2712,3=49.84, p<0.001; post-hoc Tukey adjusted ps<0.0125). A third participant group (“Control group”) did not receive stimulation during training but wore the tVNS electrodes and performed the staircase procedure to enable participant blinding. After six training blocks, participants completed a Generalization block in which they categorized new category exemplars produced by novel speakers. In this block, they did not receive tVNS or corrective feedback.

Effects of tVNS on speech category learning. First, the effects of training in the Control group receiving no stimulation were assessed. A mixed-effects model analysis was conduced with a binomial logit link. The dependent variable was the trial-by-trial response outcomes (correct vs. incorrect) of every participant in each group. It was found that a significant effect of trial for the Control group (β=0.006, z=10.57, p<0.0001; FIG. 2). This result demonstrates that training was effective in the absence of stimulation.

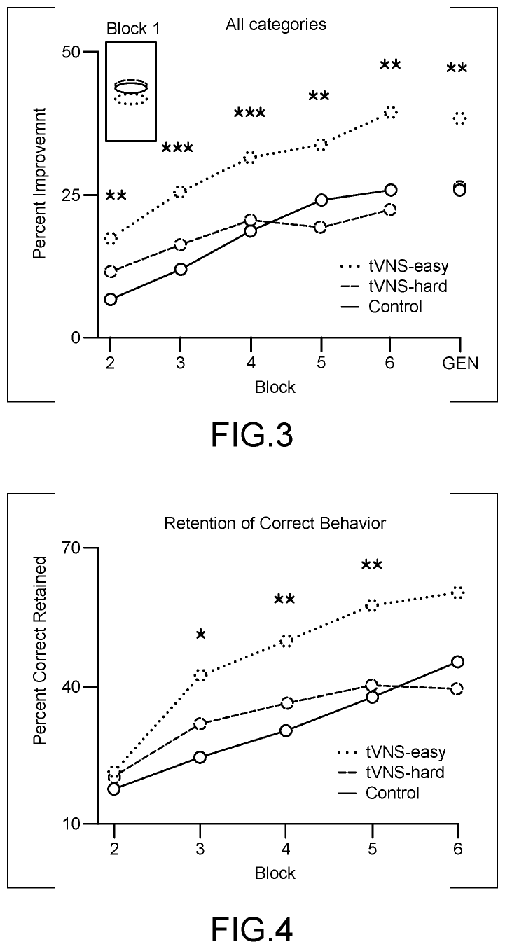

Next, the central hypothesis that pairing tVNS with specific tone categories would enhance learning was tested. To assess this hypothesis, the group-by-trial interactions in the logit mixed-effects model introduced above (Control group=reference level) was investigated. A positive and significant effect for the tVNS-easy group was observed (β=0.002, z=2.36, p=0.018; FIG. 3). This result indicates that the tVNS-easy group exhibited a better trial-by-trial improvement than the Control group. Notably, by the third block the tVNS-easy group already improved their Block 1 accuracy as much as the Control group did in the last training block (˜26% improvement). These results demonstrate that participants learned faster when tVNS was paired with the tone categories that were easier-to-learn (Tone 1 and Tone 3).

In contrast, the group-by-trial interaction for the tVNS-hard group was not as significant (β=−0.001, z=−1.83, p=0.066), indicating tVNS did not as effectively enhance learning when it was paired with the categories that were more difficult to learn (Tone 2 and Tone 4).

Next, it was asked whether tVNS improved categorization accuracy in the Generalization block, where participants categorized new speech exemplars without receiving any stimulation or corrective feedback. A logit mixed-effects model was fit with individual trial-by-block response outcomes in the Generalization block and Block 1. Block 1 was used as reference level to account for individual differences in baseline categorization performance. Block 1 was used to rule out group differences in baseline categorization performance at the onset of training. A positive and significant group-by-block interaction was found, highest for the tVNS-easy group (tVNS-easy: β=0.1, z=3.07, p=0.002; tVNS-hard: β=0.006, z=0.19, p=0.84; FIG. 3). This result indicates that the learning enhancement found in the tVNS-easy group during the training phase transferred to new category exemplars unpaired with stimulation in the generalization phase.

During the training phase (Blocks 1-6), the learning enhancement exhibited by tVNS-easy group was specific to the easier-to-learn categories. However, in the Generalization block, the advantage of the tVNS-easy group over the Control group was larger for harder-to-learn categories. This finding could be due to the confluence of two factors. First, it could be argued that, during the training phase, the Control group also improved the recognition of easier-to-learn categories (relative to harder-to-learn categories). Thus, the initial advantage the tVNS-easy group over the Control group with respect to these categories may have attenuated by the end of the task. Additionally, by the end of the task, the tVNS-easy group may have benefited from a smaller number of false positives for easier-to-learn categories and thus a smaller number of harder-to-learn exemplars miscategorized as easier-to-learn categories. To assess this hypothesis, we examined the number of false positives for easier-to-learn categories in each group. We found that the tVNS-easy group exhibited a larger reduction of these false positives over time.

Every participant completed an additional perceptual identification task before the training session and no significant group differences were found in perceptual acuity (one-way ANOVA: F35.2=0.89, p=0.41). This result indicates that the learning enhancement exhibited by the tVNS-easy group cannot be attributed to group differences in pre-training identification skills that are relevant to succeed in the speech category training task.

Retention of correct stimulus-response associations. It was assessed whether tVNS enhanced the retention of correct categorization trials between blocks. Specifically, the extent to which tVNS increased the percentage of categorization trials that were correctly categorized on block n and on block n−1 was examined. A linear mixed-effects model was fit with the individual percentages of stimulus trials that were correctly categorized in the current and previous block, starting at Block 2. The interaction for the tVNS-easy group was significant for all blocks but the last one (Block 3: β=13.95, z=2.42, p=0.016; Block 4: β=15.52, z=2.69, p=0.007; Block 5: β=16.04, z=2.78, p=0.006; Block 6: β=11.35, z=1.97, p=0.0505, FIG. 4). This result indicates that, when tVNS was paired with easier-to-learn categories, participants retained a larger proportion of correct categorization responses between most training blocks.

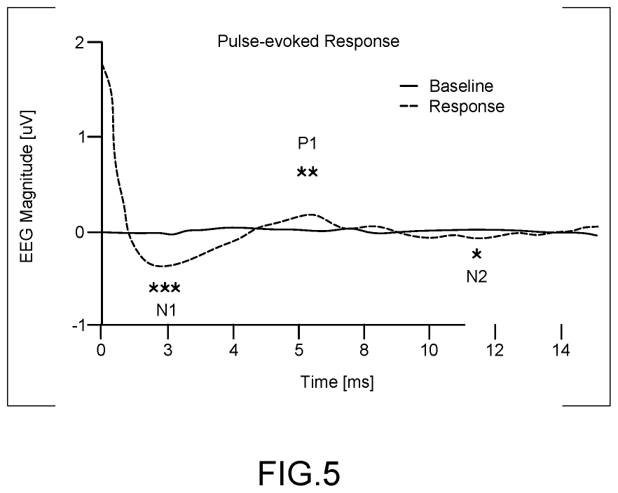

Sub-perceptual threshold vagus nerve engagement. Most previous work with tVNS has used stimulation intensities just below levels of participant discomfort, but above individual perceptual thresholds. Here, in contrast, stimulation below perceptual thresholds was used to allow participant blinding and this resulted in stimulation intensities that were several mA lower than what has been used previously in non-invasive work. Since the vagal evoked potentials reported in the literature arise at brainstem latencies (<15 ms), we collected brainstem electrophysiological responses to tVNS pulses during the training task. After removing stimulation artifacts and averaging the signals within a 15 ms window time-locked to the offset of the tVNS pulse, it was found that three tVNS pulse-evoked brainstem components with peak magnitudes significantly were different from the pre-pulse baseline magnitude: N1 (M=−1.07 μV, t40=−2.96, p=0.0051), P1 (M=0.27 μV, t40=4.75, p<0.001), and N2 (M=−0.15 μV, t34=−5.77, p<0.01). The latencies of these peaks, between 2 and 15 ms (FIG. 5), were consistent with those reported in prior tVNS work using above-threshold stimulation. Together, these results demonstrate that sub-perceptual threshold stimulation causes changes in brainstem electrophysiology that are consistent with peripheral nerve engagement.

Next was examined the extent to which the auditory sensory representation of Tone 1 and Tone 2 became more distinct from each other after the tVNS session. A machine learning classifier was implemented to decode Mandarin tone categories (Tone 1 and Tone 2) from FFRs collected before and after the tVNS session and the percentage of FFRs that were incorrectly classified were used to score the degree of confusion between the sensory representations of Tone 1 and Tone 2. Consistently with the results for FFR quality, this result indicates that the tVNS session did not have a significant impact in the sensory representation of non-native pitch.

DISCUSSION The results demonstrate that When tVNS was paired with the speech stimulus, participants performed significantly better than those who did not receive stimulation. For example, participants who received stimulation paired with Tone 1 and Tone 3 learned correct stimulus-response associations faster with accuracy differences emerging immediately after the first block (=40 trials) and retained a greater proportion of these correct associations between blocks. Crucially, this group-specific learning improvement also generalized to new speech category exemplars presented without accompanying stimulation and corrective feedback. These results demonstrate that tVNS will accelerate speech perceptual learning in humans in a highly specific manner. Overall, the results of the present study demonstrate that non-invasive, sub-perceptual threshold VNS can selectively enhance learning of complex, behaviorally relevant speech categories in adult humans.

The results indicate that the behavioral changes we observed are not due to fundamental changes to the sensory representation of fine-grained stimulus properties, but instead resulted from processes related to the adjustment of the functional mapping between broad representations of stimulus signals and abstract categories. Notably, relatively stronger peaks were found in the pulse-evoked brainstem response when subjects were stimulated with unextreme intensity currents between 1 mA and 2 mA.

These findings provide further evidence that peripheral neuromodulation is be a useful tool for augmenting behavioral and perceptual paradigms, including higher-level cognitive tasks such as speech sound learning. Together with rigorously tested training paradigms, tVNS will allow adults, who lack the neural plasticity characteristic of early childhood, to achieve substantially better outcomes in challenging tasks like learning a new language.

METHODS 36 adult native speakers of English who were unfamiliar with Mandarin Chinese were used as subject. Stimuli consisted of five Mandarin Chinese syllables (/bu/, /di/, /lu/, /ma/, and/mi/), pronounced by four native speakers of Mandarin Chinese (two females). The speakers pronounced each syllable four times, each with a different Mandarin Chinese tone, resulting in a total of 80 speech stimuli (5 syllables×4 talkers×4 tones). During the training part, half of the stimuli (N=40; two talkers) were presented in six blocks where each stimulus was played once per block. Participants indicated the tone category on each trial via button press on a keyboard (none of the buttons visually indicated pitch). Immediately following the button press, they were given feedback via visual (“Correct”/“Wrong”) text on a computer screen for 1 second. Immediately following the sixth training block, participants completed a Generalization block. In this block, they categorized the other half of the stimuli (N=40), consisting of the same syllables pronounced by two new talkers. Participants did not receive feedback or stimulation in this block. To avoid physical interference with the stimulation electrodes placed on the left ear (see “Electrical stimulation procedure” in the Methods), the audio was delivered monaurally through the right ear with an insert earphone.

To stimulate the vagus nerve non-invasively, the cymba concha and cymba cavum of the outer ear were targeted, which have been shown previously to be innervated by the auricular branch of the vagus nerve. Current was delivered transcutaneously to these sites at amplitudes below each participant's perceptual threshold. Sub-threshold stimulation avoids evoking somatosensory responses that alert participants to the timing of stimulation. The participant's left ear was first cleaned with alcohol and abrasive gel using a cotton swab. Silicon putty was then molded to the shape of the participant's ear. Two Ag—AgCl disc electrodes (4 mm diameter) were embedded in the putty at areas corresponding to the cymba concha (anode) and cymba cavum (cathode) and covered with a salt-free conductive gel. The mold was reinserted into the ear and pressed into place. Electrical stimulation was generated with a STMISOLA™ Constant Current Isolated Linear Stimulator (Biopac Systems, Goleta, Calif., US). Stimulation waveforms consisted of 15 biphasic square-wave pulses (150 μs pulse width) delivered at a rate of 25 Hz with an amplitude no higher than 3 mA. The biphasic waveforms were generated using Matlab (Mathworks, v. 2017a) and transmitted to the stimulator via a USB-6211 DAQ card (National Instruments, Austin Tex., US).

Before the speech training session, a 0.1 mA-up/0.3 mA-down staircase procedure was used to to identify the perceptual threshold in every participant. The threshold was calculated as the average stimulation amplitude after eight reversals. In the speech training session, stimulation was delivered with a pulse amplitude of 0.2 mA below each participant's perceptual threshold. A two-sample t-test revealed no significant differences in pulse amplitude (t22=1.26; p=0.21) between the two participant groups targeted with stimulation (tVNS-hard: M=1.67 mA, SD=0.79 mA; tVNS-easy: M=1.24 mA, SD=0.88 mA). The pulse train began approximately 300 ms prior to the onset of the auditory stimulus and continued for 250 ms through the stimulus, approximately half of the auditory stimulus delivery period.

To assess sub-threshold vagal evoked potentials, EEGs were recorded during the training session from all participants receiving stimulation. EEGs were collected with three Ag— AgCl scalp electrodes (impedance<5 kΩ) connected to a preamplifier (50 dB gain) from the vertex (active), left mastoid (ground) and right mastoid (reference). They were off-line band-pass filtered with a zero-phase second-order Butterworth filter roughly reflecting the phase-locking limitations of neurons in the brainstem (80 Hz-1 kHz). Each tVNS pulse left a characteristic square-wave artifact in the EEG. These artifacts were used to estimate the onset and offset of each tVNS pulse by cross-correlating a template of the pulse artifact with the EEG. Predicted and observed pulse markers were visually inspected for validation. To avoid ringing artifacts caused by the interaction of pulse artifacts with the band-pass filter, all pulse artifacts were removed before filtering the signal and Matlab function fillgaps.m was used reconstruct the gaps from nearby values (2 ms both sides the gap). Participant responses elicited three clear evoked potentials peaking at approximately 2 ms (N1), 6 ms (P1), and 11 ms (N2) after the pulse offset (FIG. 5).

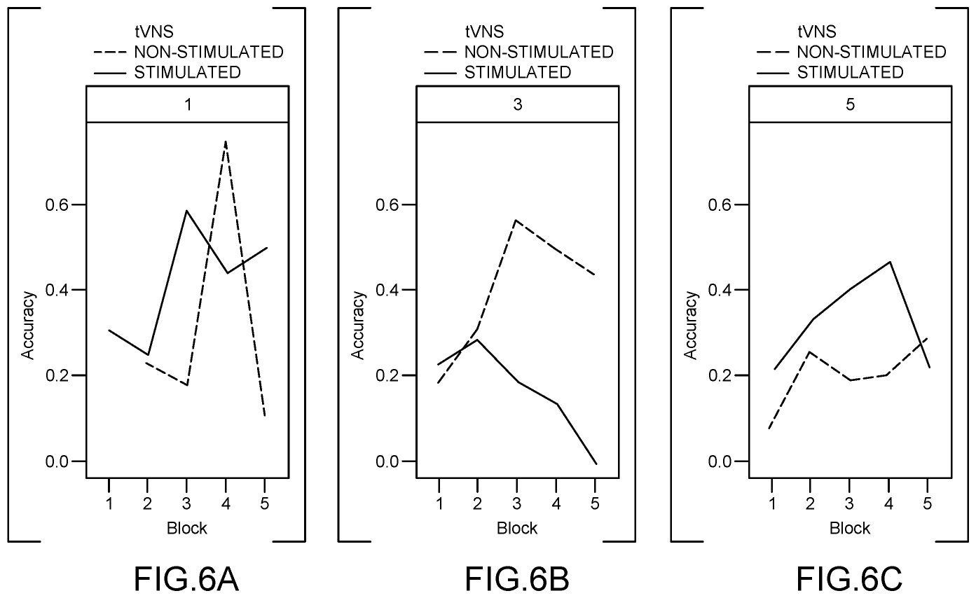

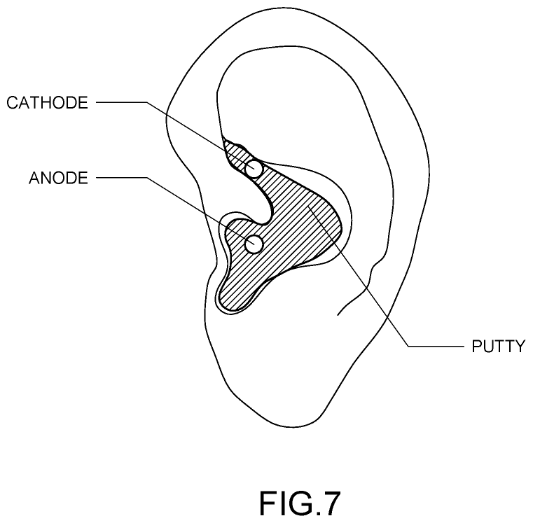

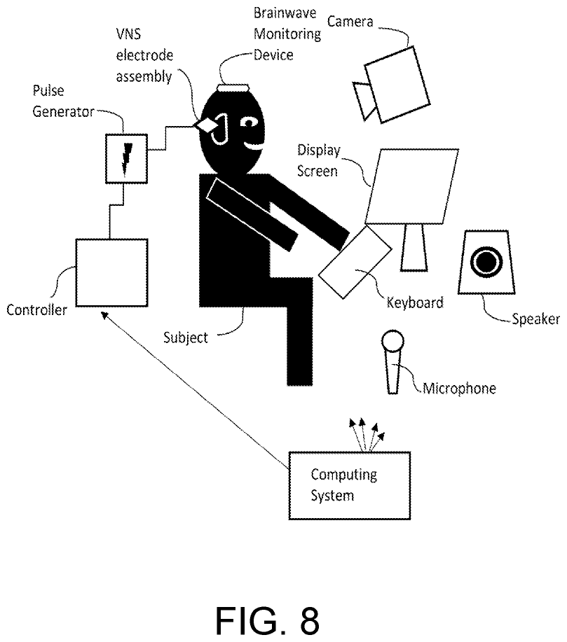

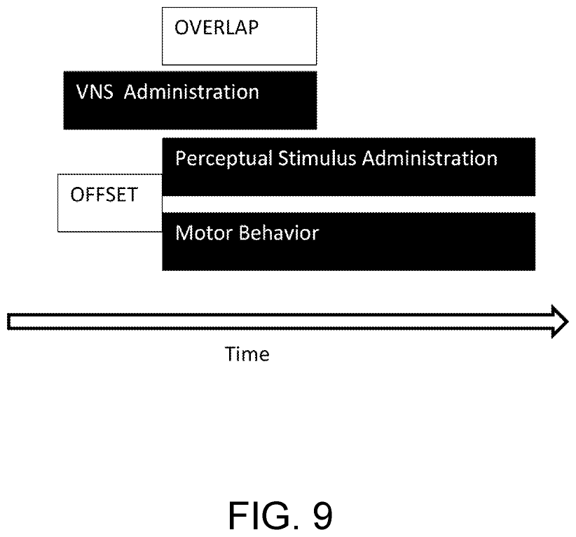

Example 2. Word Learning Task. FIGS. 6A, 6B, and 6C Depict Performance for three subjects in a word learning task where native English speakers were tasked with associating Mandarin words with objects. Subjects were administered playback of a recorded Mandarin word, then were visually presented with three definitions on a screen and asked to pick the correct one. Each block represents a set of 32 trials. Two of the three subjects (FIG. 6A and FIG. 6C) showed enhanced performance when stimuli were temporally paired with subperceptual tVNS administration. One subject (FIG. 6B) did not show improvement.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

What is claimed is:

1. A method of enhancing the efficacy a behavioral therapy in a subject,
wherein the behavioral therapy comprises a perceptual or motor behavior by the subject;
the method comprising the administration to the subject of vagus nerve stimulation;
wherein the vagus nerve stimulation is administered in a temporal alignment with the motor or perceptual behavior that is selected to enhance the efficacy of the behavioral therapy.

2. The method of claim 1, wherein
the subject is selected from a student; a person with a cognitive deficit or impairment; and an aged subject.

3. The method of claim 2, wherein
the subject is a person with a cognitive deficit or impairment, comprising a subject with surgical trauma, traumatic brain injury, neurodegeneration, brain cancer, epilepsy, or ischemia.

4. The method of claim 1, wherein
the behavioral therapy comprises the administration to the subject of a perceptual stimulus; and wherein the vagus nerve stimulation is administered in a temporal alignment with the administration to the subject of the perceptual stimulus that is selected to enhance the efficacy of the behavioral therapy.

5. The method of claim 4, wherein
the behavioral therapy comprises learning, memorization, or cognitive repair.

6. The method of claim 5, wherein
the behavioral therapy comprises learning of a non-native language.

7. The method of claim 5, wherein
the behavioral therapy comprises cognitive repair of a speech disorder or impairment.

8. The method of claim 4, wherein
the perceptual stimulus comprises an auditory stimulus.

9. The method of claim 8, wherein
the auditory stimulus comprises phonemes, syllables, or words of a non-native language.

10. The method of claim 1, wherein
the perceptual stimulus comprises a visual stimulus.

11. The method of claim 1, wherein
the perceptual stimulus comprises a tactile stimulus.

12. The method of claim 1, wherein
the behavioral therapy comprises the performance of a motor behavior by the subject; and
wherein the vagus nerve stimulation is administered in a temporal alignment with the performance of the motor behavior by the subject that is selected to enhance the efficacy of the behavioral therapy.

13. The method of claim 12, wherein
the motor task is selected from operating a device, playing a musical instrument, or a sports movement.

14. The method of claim 12, wherein
the motor task comprises movements associated with voicing a sound, phoneme, word or phrase.

15. The method of claim 1, wherein
the VNS is administered by an invasive VNS system.

16. The method of claim 1, wherein
the VNS is administered by a transcutaneous VNS system.

17. The method of claim 16, wherein
the VNS system comprises a system for stimulation of the auricular vagus nerve.

18. The method of claim 17, wherein
the system for stimulation of the auricular vagus nerve comprises one or more electrodes in contact with the cymba concha, cymba cavum, tragus, or ear canal.

19. The method of claim 1, wherein
the VNS is administered in a biphasic square wave.

20. The method of claim 19, wherein
the biphasic square wave comprises a pulse width of 50-200 μs.

21. The method of claim 1, wherein
the VNS is administered at an intensity of 0.1 to 5.0 mA.

22. The method of claim 1, wherein
the VNS is administered at a frequency of 10-300 Hz.

23. The method of claim 1, wherein
the VNS is administered at a subperceptual dosage.

24. The method of claim 23, wherein
the subperceptual dosage is administered at an intensity 0.1-2.5 mA below the measured perceptual threshold of the subject.

25. The method of claim 1, wherein
the VNS dosage is administered as a subperceptual transcutaneous VNS dosage comprising 10-20 biphasic square-wave pulses, having with a pulse width of 100-200 μs, applied at a frequency of 20-30 Hz, at an intensity between 0.1 mA and 5.0 mA, selected to be 0.1-2.5 mA below the participant's perceptual threshold.

26. The method of claim 1, wherein
the behavioral therapy comprises the administration of a perceptual stimulus to the subject; and
wherein the selected temporal alignment of the VNS administration and the administration of the administration of the perceptual stimulus comprises commencement of VNS administration prior to the commencement of the administration of the perceptual stimulus to the subject by a selected time interval.

27. The method of claim 26, wherein
the selected time interval is 10-2,000 ms prior to the commencement of the administration of the perceptual stimulus.

28. The method of claim 27, wherein
the selected time interval is 100-500 ms prior to the commencement of the administration of the perceptual stimulus.

29. The method of claim 26, wherein
the completion of VNS administration comprises an endpoint during the administration of the perceptual stimulus.

30. The method of claim 29, wherein
the completion of the VNS administration is 10-500 ms after commencement of administration of the perceptual stimulus.

31. The method of claim 29, wherein
the completion of the VNS administration comprises an endpoint at 25-75% of the duration of the administration of the perceptual stimulus.

32. The method of claim 1, wherein
the behavioral therapy comprises the performance of a motor behavior by the subject; and
wherein the commencement of VNS administration is prior to the commencement of the performance of the motor behavior by the subject.

33. The method of claim 32, wherein
the selected time interval is 10-2,000 ms prior to the commencement of the performance of the motor behavior by the subject.

34. The method of claim 33, wherein
the selected time interval is 100-500 ms prior to commencement of the performance of the motor behavior by the subject.

35. The method of claim 32, wherein
the completion of VNS administration comprises an endpoint during the performance of the motor behavior by the subject.

36. The method of claim 35, wherein
the completion of the VNS administration is 10-500 ms after the commencement of the performance of the motor behavior by the subject.

37. The method of claim 35, wherein
the completion of the VNS administration comprises an endpoint at 25-75% of the duration of performance of the motor behavior by the subject.

38. The method of claim 1, wherein
the VNS dosage is adjusted based on evaluation of the subject's performance or attainment of a target neural state.

39. The method of claim 38, wherein
the evaluation of the subject's performance is assessed by an automated means.

40. The method of claim 39, wherein
the attainment of a target neural state is assessed by EMG, EEG, or ECoG.

41. The method of claim 38, wherein
the VNS dosage is not administered if the subject does not successfully perform a selected outcome or achieve a selected neural state.

42. A system for use in a method of enhancing the efficacy of a behavioral therapy,
wherein the method encompasses the performance of a method selected from any of claims 1-41,
the system comprising:
one or more devices for the administration of VNS to the subject; and
one or more control elements that time the administration of VNS to the subject to the perceptual behavior or motor behavior with a selected temporal alignment.

43. The system of claim 42, wherein
the system comprises:
one or more devices for the administration of the selected perceptual stimulus to a subject;
one or more devices for the administration of a VNS dosage to the subject; and
one or more control elements time the VNS administration window to the administration of the perceptual stimulus by a selected temporal alignment.

44. The system of claim 42, wherein
the system comprises:
one or more devices for detecting, or predicting the timing of, the initiation of a motor behavior;
one or more devices for the administration of a VNS dosage the subject; and
one or more control elements that time the VNS administration to the detected or predicted timing of the motor behavior by a selected temporal alignment.

45. A non-transitory storage medium comprising a set of computer-readable instructions which direct hardware elements to carry out the steps of the methods of any of claims 1-41.
